(19) United States
(12) Plant Patent Application Publication
(10) Pub. No.: US 2014/0317803 P1
(43) Pub. Date: Oct. 23, 2014

(54) Phalaenopsis plant named "Phal. Younghome Venus"

(50) ;

(71) Applicant: YOUNG HOME ORCHID CO., LTD., Pingtung (TW)

(72) Inventor: Chun-Teng Lin, Pingtung (TW)

(73) Assignee: Young Home Orchid CO., LTD., Pingtung (TW)

(21) Appl. No.: 13/986,264

(22) Filed: Apr. 17, 2013

(51) Int. Cl. A01H 5/00

(52) U.S. Cl. PLT/311

(57) ABSTRACT

Phal. Younghome Venus has not been observed under all possible environmental conditions. The phenotype may vary significantly with variations in environment such as temperature and light intensity, without however, any change in genotype. In the following description, color reference are made to The Royal Horticultural Society (R.H.S.) Colour Chart, 2007, fifth editon, except where terms of ordinary dictionary significance are used.

Dtps. Younghome Venus used for aforementioned photographs and following detailed botanical description were 11 months old and grown in 12 cm containers in Pingtung, Taiwan, in a controlled green house with day temperature about 26˜29° C., night temperature about 18˜20° C., and light level about 15,000˜20,000 lux. The photographs and detailed botanical description were taken during the winter.

### FIELD OF THE INVENTION

Botanical classification: Phalaenopsis

Variety denomination: Phal. Younghome Venus

### BACKGROUND OF THE INVENTION

The present invention comprises a new and distinct cultivar of Phalaenopsis Orchid, and hereinafter referred to by the cultivar name, Phal. Younghome Venus.

The new cultivar is a product of a planned breeding program conducted by the Inventor in Pingtung, Taiwan. The objustive of the breeding program is to create new uniform pot-type Phalaenopsis Orchid cultivars having attractive flower coloration.

Asexual propagation by tissue culture in a laboratory in Pingtung, Taiwan has been used to increase the number of plants for evaluation and has demonstrated that the unique combination of characteristics as herein disclosed for the new Phalaenopsis Orchid are firmly fixed and are retained through successive generations of asexual reproduction.

### SUMMARY OF THE INVENTION

The following traits have been repeatedly observed and are determined to be basic characteristics of new cultivar which in combination distinguish this Phalaenopsis Orchid as a new and distinct cultivar:
1. White flower with red purple color shaded around the column.
2. Freely flowering habit.
3. Upright, freely branching and sturdy flowering stem.
4. Strong growth ability and disease resistance.

Plants of the new cultivar differ primarily from plants of the parent cultivars in flower color. Plants of Phal. Younghome Venus can be compared to the Phal. Sogo Yukidian (not patented) in the following characteristics:
1. Plants of the new Phalaenopsis orchid are smaller than plants of the cultivar Phal. Sogo Yukidian.
2. Plants of the new Phalaenopsis orchid have shorter spikes than plants of the cultivar Phal. Sogo Yukidian.
3. Plants of the new Phalaenopsis orchid have smaller flower size than plants of the cultivar Phal. Sogo Yukidian.
4. The petals for plants of the new Phalaenopsis orchid are white and red purple around the column, while plants of the cultivar Phal. Sogo Yukidian is white.
5. The new Phalaenopsis orchid have orange labellum and plants of the cultivar Phal. Sogo Yukidian is yellow.

### BRIEF DESCRIPTION OF THE DRAWINGS

Colors in the photographs may appear different from the color values that appear in the detailed botanical description which more accurately describe the new cultivar.

FIG. 1 is a side view of a plant of Phal. Younghome Venus flowering in the pot of 12 cm.

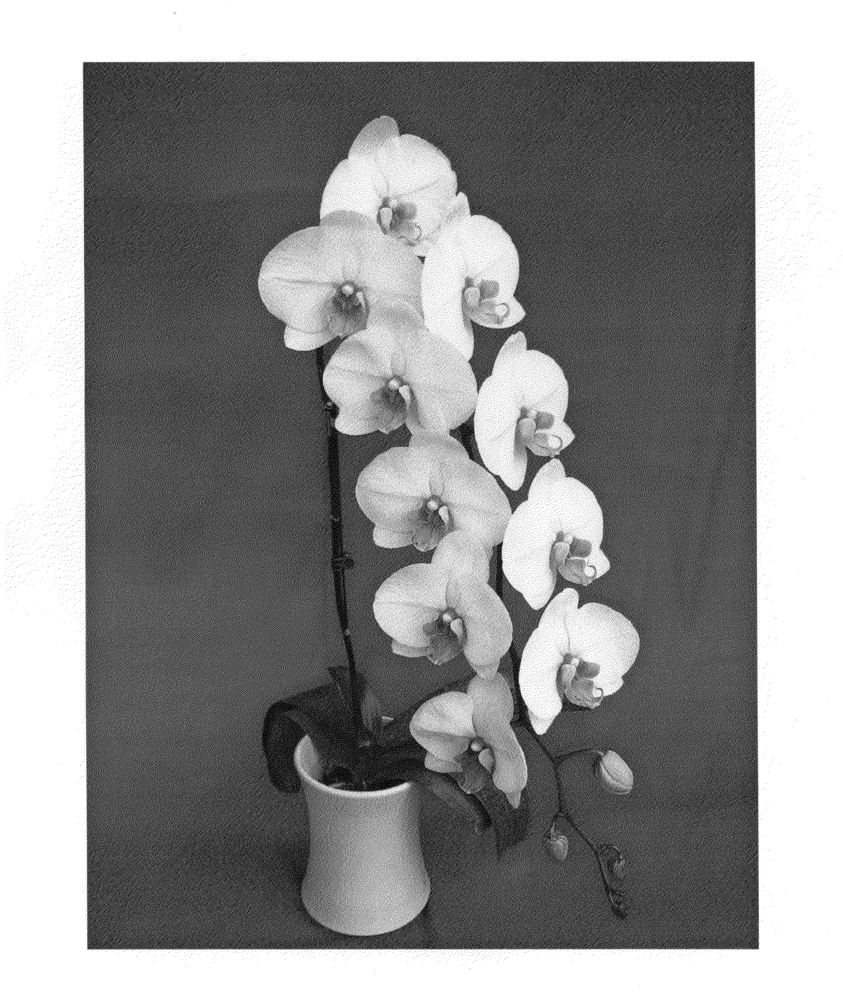

FIG. 2 is a close-up view showing the characteristics of the flower.

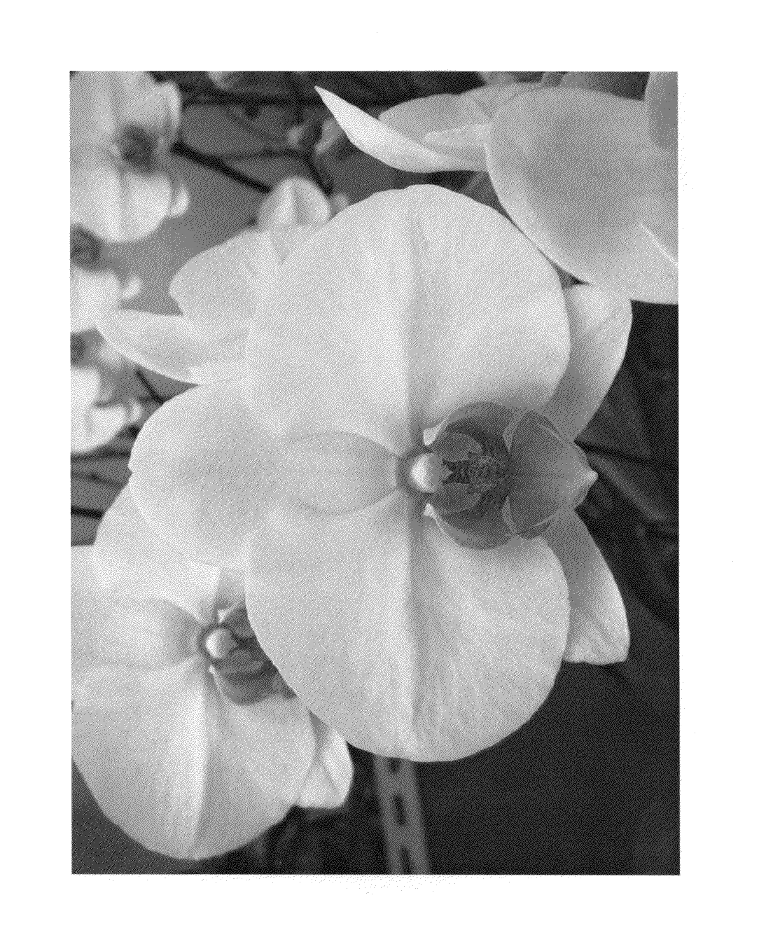

### DETAILED DESCRIPTION OF THE PREFERRED EMBODIMENTS

Phal. Younghome Venus has not been observed under all possible environmental conditions. The phenotype may vary significantly with variations in environment such as temperature and light intensity, without however, any change in genotype. In the following description, color reference are made to The Royal Horticultural Society (R.H.S.) Colour Chart, 2007, fifth editon, except where terms of ordinary dictionary significance are used.

Phal. Younghome Venus used for aforementioned photographs and following detailed botanical description were 11 months old and grown in 12 cm containers in Pingtung, Taiwan, in a controlled green house with day temperature about 26˜29° C., night temperature about 18˜20° C., and light level about 15,000˜20,000 lux. The photographs and detailed botanical description were taken during the winter.
Parentage:
   Seed.—Phal. Hsinying Mount.
   Pollen.—Phal. Maki Watanabe.
Propagation: Asexual propagation by tissue culture. (agree)
Plant description: Plant shape, Two-ranked leaves affixed to a short central stem (monopodial growth). Single flowers arranged on upright and sturdy flowering rancemes. Plant height, soil level to top of foliar plane. — About 13 to 15 cm. Plant height, soil level to top of inflorescences. — About 62 to 67 cm. Plant diameter — About 35 to 45 cm.
Flowers per stem: Approximately 8 to 11.
Foliage description:
   Leaf arrangement.—Alternate.
   Quantity per plant.—About 6 to 8.
   Length.—About 25 to 35 cm.
   Width.—About 6 to 8 cm.
   Shape.—elliptic, elongated.
   Apex.—oblong, obtuse.
   Base.—cuneate.
   Margin.—entire.
   Aspect.—mostly flat and folded upward from the midrib.
   Texture, upper and lower surfaces.—leathery, thick.
   Venation.—parallel, veins are sunken within the lamina.
   Color.—Upper surface — green(RHS N137C) with purple (RHS N79B) discoloration.
   Color.—Lower surface — green(RHS N137C) with purple (RHS N79B) discoloration.
Flower description: Inflorescence type — Compound raceme. Flower type — Single zygomorphic flowers arranged in racemes. Flowers roughly pentagonal in shape. Flower arrangement — compound alternate Flowering stems — Upright, freely branching and sturdy. Flowering habit — Plants freely flowering; plants typically produce 1 to 2 branched flowering stems with at least 8 to 11 flowers each. Fragrance — negative. Natural flowering season — From January to April in Taiwan. Post-production longevity — Plants of the ‘new Phalaenopsis orchid’ maintain good leaf and flower substance for about 1.5 to 2 months on the plant under interior environmental conditions. Inflorescence length — About 12 to 34 cm. Inflorescence diameter — About 16 to 20 cm. Flower diameter — About 10 to 11 cm. Flower depth — About 3 cm. Flower bud Flower bud shape — orbicular. Flower bud length — About 2 cm. Flower bud diameter. — About 1 cm. Flower bud color — yellow-green(RHS146B).
Petals: Number of petals — 2 per flower. Length — About 6.5 cm. Diameter — About 5 cm. Shape — obovate. Apex — rounded. Base — attenuate and fused with the column. Margin — attenuate and fused with the column. Texture, upper and lower surfaces — smooth. Petal main color — white (RHS NN155D). Petal pattern color — red purple (RHS N74B) around the column.
Labellum: Length — About 2.3 cm. Diameter — About 2 cm. Shape — Deeply three-lobed with two prominent callosities on the upper surface at the central junction of the lateral lobe and the base of the middle lobe. The shape of the middle lobe is rhombic with upend hook shape and whiskers at the apex. Base color of the apical lobe — light orange (RHS 24B). Tip color of the apical lobe — orange(RHS 24A).
Sepals: Quantity — 3 per flower. Dorsal sepal length — About 5.5 cm. Lateral sepal length — About 5 cm. Dorsal sepal diameter — About 4 cm. Lateral sepal diameter — About 3 cm. Shape — ovate. Apex — round. Base — attenuate and fused with the column. Margin — entire. Texture, upper and lower surfaces — velvety and smooth. Dorsal sepal main color — white (RHS NN155D). Dorsal sepal pattern color — red purple (RHS N74B) around the column. Lateral sepal main color — white (RHS NN155D). Lateral sepal pattern color — green-yellow (RHS 1D).
Peduncles: Length — About 30 to 36 cm. Diameter — About 0.6 cm. Aspect — upright. Strength — strong. Texture — smooth, glabrous. Color — green(RHS N137A).
Pedicels: Length — About 3 cm. Diameter — About 3 mm. Aspect — About 75° from vertical, two spikes about 55° from vertical. Strength — strong, sturdy. Texture — smooth, glabrous. Color — green(RHS N137A). Color towards the base — green(RHS N137A).
Reproductive organs:
   Column.—Length — About 0.7 cm. Diameter — About 6 mm. Color — white (RHS NN155D) in the front and red purple (RHS N74B) in the back. Color towards to apex — white (RHS NN155D).
   Pollinia.—Quantity of pollen masses — 2. Diameter — About 2 mm. Color — yellow-orange (RHS23C).
   Ovary.—Length — About 5 mm. Diameter — About 4.5 mm. Color — light orange (RHS 24B) base with grayed-orange(RHS 174A) spotted.
   Root.—It takes 14 days for plants growing in tissue culture to initiate roots. Diameter — About 6 mm. Color — RHS 138A.
Disease/pest resistance: Strong disease resistance, but no specific resistance or susceptibility to pests has been observed.
Temperature tolerance: The plant can be tolerant to temperature from 16˜35° C. The flower can be tolerant from 20˜28° C.

What is claimed is:

1. A new and distinct cultivar of Phalaenopsis orchid plant named ‘Phal. Younghome Venus’ as illustrated and described.
